SEQUENCE LISTING (9 sequences)

SEQ ID NO: 1 - DNA, 4791 bp, Plasmid pSCp36
  misc_feature (1939)..(1045): Kanamycin resistance, reverse complementary
tcttccgcttcctcgctcactgactcgctgcgctcggtcgttcggctgcggcgagcggtatcagctcactcaaaggcggtaatacggttatccacagaatcaggggataacgcaggaaagaacatgtctcgggaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggtctcctcccggatcctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagaagctttattgcggtagtttatcacagttaaattgctaacgcagtcagtgctcgagcaggtaagtatcaaggttacaagacaggtttaaggaggccaatagaaactgggcttgtcgagacagagaagactcttgcgtttctgataggcacctattggtcttactgacatccactttgcctttctctccacaggggtaccatgaactacgagggtcacctgaagggccaccgcggatgggtcacctccctggcctgcccgcagcaggcggggtcgtacatcaaggtggtgtcgacgtcgcgcgatggcacggccatctcgtggaaagccaaccccgaccgccacagcgtggacagcgactacggtctgccgagccaccgcctcgagggccacaccggcttcgtgtcgtgtgtgtcgctggcccacgccaccgactacgcgctgaccgcgtcctgggaccgctccatccgcatgtgggacctgcgcaatggccagtgccagcgcaagttcctgaagcacaccaaggacgtgctcgccgtcgccttctcgccggacgaccgcctgatcgtgtccgcgggccgcgacaacgtgatccgcgtgtggaacgtggcgggcgagtgcatgcacgagttcctgcgcgacggccacgaggactgggtgagcagcatctgtttctcgccgtcgctggagcatccgatcgtggtgtccggcagctgggacaacaccatcaaggtatggaacgtgaacgggggcaagtgtgagcgcacgctcaagggccacagcaactacgtgtccacggtgacggtgtcgccagacgggtcgctgtgcgcgtccggcggcaaggacggcgcggcgctgctgtgggacctgagcaccggcgagcagctgttcaagatcaacgtggagtcgcccatcaaccagatcgccttctcgcccaaccgcttctggatgtgcgtcgcgacggagaggtccctgtccgtgtacgacctggagagcaaggctgtgattgcggagctgacgccggacggcgcgaagccgtccgagtgcatctccattgcctggtccgccgacggcaacactctgtactccggtcacaaggacaacctgatccgcgtgtggtccatctccgacgccgagtaagagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagaattcagggggagacccaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 2 - protein, 11 aa, Human immunodeficiency virus type 1, TAT peptide
YGRKKRRQRRR

SEQ ID NO: 3 - protein, 7 aa, Simian virus 40, NLS peptide
PKKKRKV

SEQ ID NO: 4 - DNA, 20 bp, Unknown
  ODN 1
gggagtccagttttctggac

SEQ ID NO: 5 - DNA, 20 bp, Unknown
  ODN 2
aggggtccagttttctggac

SEQ ID NO: 6 - DNA, 33 bp, Unknown
  1. PCR Primer left
ttatatggtaccatgaacatacgagggtcacct

SEQ ID NO: 7 - DNA, 42 bp, Unknown
  1. PCR Primer right
ttatatgagctcagaagacacggacagggacctcttccgtcg

SEQ ID NO: 8 - DNA, 33 bp, Unknown; identical to SEQ ID NO: 6
  2. PCR Primer left

SEQ ID NO: 9 - DNA, 33 bp, Unknown
  2. PCR Primer right
ttatatgagctcttactcggccgtcggagatgg

(19) United States
(12) Patent Application Publication
(10) Pub. No.: US 2004/0235772 A1
(43) Pub. Date: Nov. 25, 2004

(54) DNA expression construct for the treatment of infections with leishmaniasis

(76) Inventors: Laura Fuertes-Lopez, Madrid (ES); Marcos Timon-Jimenez, San Lorenzo De El Escorial (ES)

Correspondence Address: NILS H. LJUNGMAN, ESQUIRE, NILS H. LJUNGMAN & ASSOCIATES, P.O. BOX 130, GREENSBURG, PA, 15601-0130, US

(21) Appl. No.: 10/816,591

(22) Filed: Apr. 1, 2004

Related U.S. Application Data
(63) Continuation-in-part of application No. PCT/DE02/03799, filed on Oct. 2, 2002.

(30) Foreign Application Priority Data
Oct. 2, 2001 (DE) 101 48 732.0
Nov. 12, 2001 (DE) 101 56 679.4

(51) Int. Cl. A61K 48/00; C07H 21/04; C12N 15/74

(52) U.S. Cl. 514/44; 435/320.1; 536/23.7

(57) ABSTRACT

DNA expression construct for the treatment of infections with leishmania, as well as a corresponding vaccine. This vaccine serves to immunise against leishmaniasis, where in particular the immunogenic p36 LACK antigen is used to elicit an immune response. As a gene shuttle, a linear double stranded, covalently closed expression cassette is employed. The gene expression construct can be attached to an oligopeptide to increase transfection efficacy. The abstract of the disclosure is submitted herewith as required by 37 C.F.R. §1.72(b). As stated in 37 C.F.R. §1.72(b): A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims. Therefore, any statements made relating to the abstract are not intended to limit the claims in any manner and should not be interpreted as limiting the claims in any manner.

### CONTINUING APPLICATION DATA

This application is a Continuation-In-Part application of International Patent Application No. PCT/DE02/03799, filed on Oct. 2, 2002, which claims priority from Federal Republic of Germany Patent Application Nos. 101 48 732.0, filed on Oct. 2, 2001, and 101 56 679.4, filed on Nov. 12, 2001. International Patent Application No. PCT/DE02/03799 was pending as of the filing date of this application. The United States was an elected state in International Patent Application No. PCT/DE02/03799.

### BACKGROUND

1. Technical Field

This application concerns a DNA expression construct for the treatment of infections with leishmania, and a corresponding vaccine.

Leishmania are trypanosmatide flagellates of the order Kinetoplastida. They are passed on to different mammal species and humans by female blood-feeding sandflies of the species Phlebotomus and Lutzomyia. Leishmaniases are diseases with a diverse set of clinical appearances and constitute a major health problem. According to WHO estimates, about 12 million human beings are affected by the disease world-wide. About 2 to 9 percent of all HIV patients suffer from visceral leishmaniasis, making it the third most prevalent parasitic disease afflicting HIV patients.

Chemotherapy shows only a limited effect as a treatment.

Since patients who have overcome the infection develop a strong immunity against subsequent infection, the development of an effective vaccine should be possible.

2. Background Information

The principle of immunization is based on the recognition by the immune system of structures of pathogens that have been successfully fought against in the past. Two main pathways are to be distinguished: the humoral pathway, relying on the production of antibodies that are able to combat bacteria in the intracellular space, and the cellular pathway, which is based on the activity of T-lymphocytes of the immune system. T-lymphocytes are able to recognize cells that are infected by virus. The humoral immune response is also referred to as the Th2 pathway and the cellular response is called Th1 pathway. Vaccination or immunotherapy of leishmaniasis, the causative agents of which are intracellular parasites, should be possible by means of the induction of a Th1 type immune response. In the state of the art, reference is made frequently to the importance of the induction of a Th1 response for therapy or prevention of leishmaniasis (Handman et al., J Immunol 160: 3949-57, Gurunathan et al., Nature Med: 4(12): 1409-15). In order to facilitate the triggering of a type Th1 immune response, reference is made to the indispensability of the co-stimulatory cytokine IL-12 as an adjuvant (Parker et al., J. Immunol. 140: 896-902).

Different antigens were tested in various experimental vaccine protocols in mice. Balb/c mice are a good model for studying leishmaniasis. Important similarities exist with regard to the progression of the infection and the development of lesions between mice and men. The immunological reaction to this infection in mice seems to be similar to that in humans, and probably also to that in dogs (Cox, Int. J. Parasitol. 263: 1147-1157). Antigens employed were gp63 (Scott et al., J. Exp Med. 168: 1675-1684), gp46 (McMahon-Pratt et al., Infection and Immunity 61: 3351-3359), p-4 and p-8 (Scott et al., Immunology 99: 615-624) and the antigen referred to as gp36 or LACK (Gonzales-Aseguinolaza et al., Eur. J. Biochem. 259: 909-916). LACK is a 36 kDa antigen from leishmania, which is highly conserved and found in all related species of leishmania. It is expressed both in the promastigote and amastigote, the two life stages of the parasitic life cycle in the host. It has been shown in the mouse model that the Induction of a cytotoxic Th1 immune response relying on interferon gamma and secretion of IL-12 by T helper cells helps in overcoming a challenge infection, whereas the induction of an IL-4 driven Th2 helper cell response favors infection. In order to support the shifting of the immune response towards the Th1 type, Gonzalo et al. employed vaccina virus as a viral gene transfer method, and IL-12 as adjuvant. They used p36/LACK as antigen. Different vaccination protocols were assembled. The most successful vaccination protocol, primary immunization by p36 protein and secondary immunization by recombinant vaccinia virus encoding p36 and IL-12 led to an average decrease in lesions of 52% in comparison to non-vaccinated mice. Greatest degree of protection against infection was seen in animals that had the highest titer of IgG2a antibody (Gonzalo et al., Microbes and Infection: 3 (9): 701-711).

Different chemical, physical and biological methods of transfection are known in order to transfer the DNA encoding the immunogenic antigens or parts thereof.

Biological means of transfection, so-called gene shuttles, are viral vectors, plasmids or covalently closed minimalistic DNA constructs, referred to as MIDGE (MINIMALISTIC IMMUNOLOGICALLY DEFINED GENE EXPRESSION VECTORS, see EP 0 914 318 B1) in the following.

Plasmids are obtained by bacterial fermentation. Apart from the desired gene, they contain DNA necessary for their proliferation and selection, commonly resistance genes against antibiotics used in bacterial fermentation. This bacterial DNA has the disadvantage that it can contain immunostimulatory sequences (“ISS”, i.e. non-methylated cytosine-guanine dinucleotides, “CpG”). This effect is specifically undesirable in immunosuppresion (described in detail in DE 199 35 756). When using gene expression constructs on the basis of plasmid DNA, also the inherent risk of dissemination of antibiotic resistance genes exists, which seems especially irresponsible in the context of vaccination campaigns. For this reason, also the method of vaccination with eucaryotic expression vectors containing the leishmania specific p36 LACK antigen, suggested by Gurunathan et al. (J. Exp. Med., Vol 186, No. 7, (1997): 1137-1147), is very disadvantageous. The disadvantages of plasmid based expression vectors described above constitute an obstacle to their broad use in medical practice.

The gene transfer method most frequently used due to it great transfection efficiency is the use of viral vectors. Nonetheless the safety risks associated with their use are a hindrance to their broad application. It is known that a high risk exists that the host organism will mount a cytotoxic reaction to the transfected cells. The application of a high dose of an adenovirus led to the death of a patient in a clinical trial; it seems that an over-reaction of the immune system was the cause of this (Lehrman, 1999, Nature 401: 517-518). Furthermore, the reversion of an attenuated vaccine strain into a virulent strain by instability can not be excluded. Also, the viral components can be immunogenic by themselves, which leads to a decrease of their efficacy by the immune system of the patient.

Apart from these disadvantages, which originate in the deficiencies of current methods of gene transfer, it has not been possible despite all efforts to develop an effective and safe protection by vaccination against leishmania.

### OBJECT OR OBJECTS

It is the objective of at least one possible embodiment to provide a means to enable safe, effective and protective vaccination against leishmaniasis.

### SUMMARY

The objective is attained in at least one possible embodiment described herein.

At least one embodiment is based on providing a DNA expression construct for immunization of infections by leishmania, where the immunizing polynucleotide sequences are provided in the form of expression constructs that consist of covalently closed linear deoxyribonucleotide molecules comprising a linear double stranded region, where the single strands forming the double strand are linked by a short single stranded loop consisting of deoxyribonucleotides, where said double strand forming single strands only consist of the coding sequence under control of a promoter that is operable in the animal that is to be vaccinated, and a terminator sequence, and where the construct is linked to one or more peptides in order to enhance transfection efficiency (see EP 0 941 318 B1). A DNA expression construct of this kind provides for surprising effects (see below) that can not be attained by other methods (such as the vaccination protocols cited); in particular, vaccination according to at least one embodiment does not bring with it the disadvantages of eucaryotic expression vectors described above.

According to the prevention, the use of the immunogenic p36 LACK antigen for the provocation of an immune response is planned. A linear double stranded covalently closed expression cassette is used as gene transfer agent. This cassette consists of the coding sequence, the promoter and optional terminator sequences, so that the construct only contains the information necessary for the expression of the desired gene (see EP 0 941 318 B1). Furthermore it is provided by at least one embodiment that the DNA expression construct is covalently attached to an oligopeptide in order increase the efficacy of transfection, the oligopeptide preferably having a length of five to 25 amino acids and at least consisting by half of amino acids taken from the group of lysine and arginine. Of special preference is a nuclear localization sequence, preferably

the sequence PKKKRKV (proline-lysine-lysine-lysine-arginine-lysine-valine=Seq ID 3) comprising a nuclear localization signal (NLS) from the simian virus SV40. It was demonstrated for the SV40 NLS that proteins up to 465 kDa are directed towards the nucleus (Lanford et al. 1986, Cell 15; 46 (4): 575-82). This quality of the peptide was utilized here by attachment to the DNA in order to achieve an increase of gene transfer,

or the eleven amino acid T peptide fragment (YGRKKRRQRRR=Seq ID 2) of the HIV-1 gene product TAT.

Further advantageous aspects of at least one embodiment are contained in the features below. At least one embodiment is described and discussed in more detail in examples and figures below.

The surprising effect of the DNA expression construct according to at least one embodiment, and a pharmaceutical product containing such construct, is exemplified in the presentations contained in the Figures. The abbreviations signify:

| | |
|---|---|
| pMOK p36 | plasmid encoding p36 antigen |
| Mp36-NLS | MIDGE encoding p36 antigen attached to NLS Peptid |
| pMOK ctr | control plasmid encoding HBsAg |
| rVVp36 | recombinant Vaccinia Virus encoding p36 |
| phosphate | phosphate buffer as control |
| control + | positive control, sera of mice infected with L. major |
| control − | negative control, sera of untreated mice |

The above-discussed embodiments of the present invention will be described further hereinbelow. When the word “invention” or “embodiment of the invention” is used in this specification, the word “invention” or “embodiment of the invention” includes “inventions” or “embodiments of the invention”, that is the plural of “invention” or “embodiment of the invention”. By stating “invention” or “embodiment of the invention”, the Applicant does not in any way admit that the present application does not include more than one patentably and non-obviously distinct invention, and maintains that this application may include more than one patentably and non-obviously distinct invention. The Applicant hereby asserts that the disclosure of this application may include more than one invention, and, in the event that there is more than one invention, that these inventions may be patentable and non-obvious one with respect to the other.

### BRIEF DESCRIPTION OF THE DRAWINGS

Further advantageous measures are described herein below; at least one possible embodiment is described in more detail in the following by means of examples and figures. It is shown in FIG. 1: determination of the total IgG antibody titer before challenge infection with Leishmania major promastigotes. Only vaccination protocols containing a secondary immunization with recombinant vaccinia virus show a measurable antibody titer.

FIG. 2: determination of the total IgG antibody titer after challenge infection. All vaccination protocols show a measurable antibody response, whereas the highest titer of circulating antibody is provoked by MIDGE p36-NLS/MIDGE p36-NLS.

FIG. 3: ratio of the antibody isotypes IgG 2a and IgG 1 after secondary immunization and challenge infection by L. major Promastigotes. Surprisingly, NLS-coupled MIDGE provoked an immune response that was more cytotoxic in nature as read out from the antibody isotype distribution, and only marginally different from the response elicited by the regime pMOKp36.

FIG. 4: the development of lesion in a time frame of 8 weeks after challenge infection. Vaccination protocols based on MIDGE p36-NLS/MIDGE p36-NLS and pMOK p36/rVV p36 resulted in the longest protection against infection by leishmania major. Protection is apparent by slowed development of lesions. The most efficient and longest lasting protection, however is attained in the group MIDGE p36-NLS/MIDGE p36-NLS.

FIG. 5: the size of lesions after week 8. In week 8 after challenge infection, lesion size is 80% smaller in the animals vaccinated MIDGE p36-NLS/MIDGE p36-NLS compared to the non-vaccinated control group. An increase of 11% in protection against L. major could be seen in comparison with the group vaccinated with p36/rVV.

### DESCRIPTION OF EMBODIMENT OR EMBODIMENTS

It was investigated whether modification of the minimalistic expression cassettes by attaching peptides was able to change the strength or bias of the immune response. In order to increase transfection efficacy, attempts were made to covalently link different peptides and other organic molecules to the MIDGE vectors.

Thus, it was possible to demonstrate a 10 to 15-fold increase in antibody titer after intramuscular application by covalent attachment of the nuclear localization signal from SV40 virus to MIDGE encoding HBsAg after intramuscular application (Schirmbeck et al., J. Mol Med. 2001 Jun. 79 (5-6): 343-50).

In a vaccination trial in mice, different gene expression constructs, all of which encoded the antigen p36 LACK, were tested. MIDGE linked to NLS peptide (MIDGE p36-NLS), plasmid (pMOKp36) and recombinant vaccinia virus (rVVp36) was employed. In order to attain a maximal degree of protection, different vaccination regimes were designed. As relevant parameters for the clinical success of the vaccination, the growth of infection related lesions in the infected hind paw of the animal are measured. As a surrogate parameter the ratio of IgG1 and IgG2a antibody subtypes was determined. It can be generalized that a subtype ratio shifted towards IgG2a correlates with protection from infection or a significantly slowed progression of the lesions. Apart from the method of primary immunization with plasmid and secondary immunization with recombinant vaccina virus (rVV), which is known from the state of the art, it was intended to determine whether a similar protection by vaccination could also be attained by the pharmaceutical preparation according to at least one embodiment.

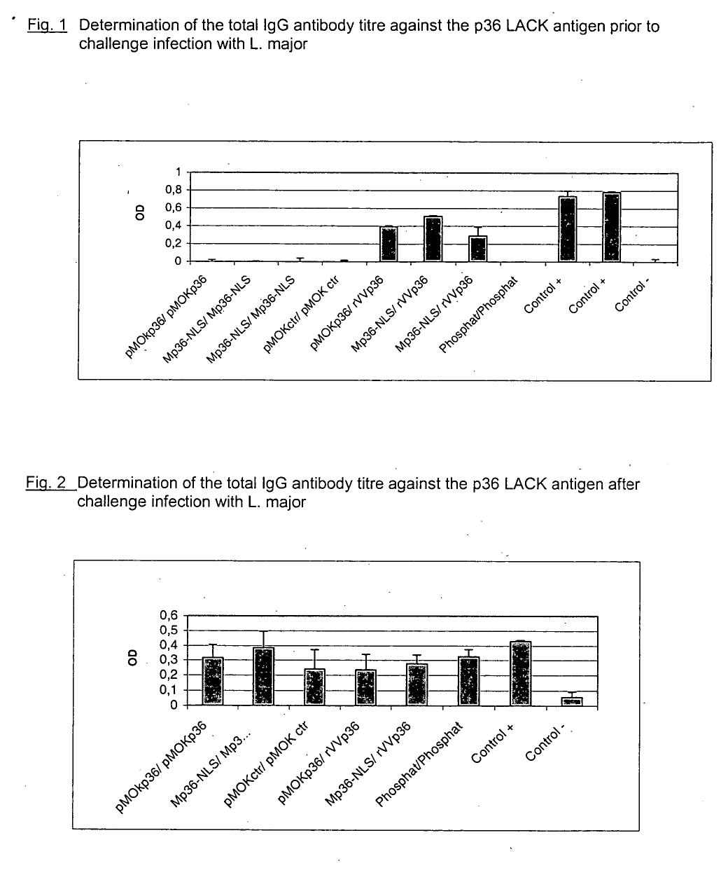

Antibody titers for total IgG were determined by ELISA as a measure for the triggering of an immune response. Only two vaccination regimes were able to elicit measurable antibody levels prior to challenge infection with leishmania major promastigotes (see FIG. 1). In both cases, recombinant vaccinia virus was used as a secondary immunization (boost). Different studies have shown that circulating antibodies alone can not be taken as an indication of a supposed protective effect. A connection between circulating antibody and protection against infection can only be determined after challenge infection. FIG. 2 shows the antibody titers after challenge infection with L. major. All vaccination regimes show measurable antibody titers, the highest titer having been achieved with MIDGE p36-NLS/MIDGE p36-NLS.

Antibody subtypes IgG1 and IgG2a were determined in order to demonstrate an eventual shift in immune response bias. Isotype distribution immunoglobulin gamma (IgG) specific for a given antigen reflects the bias of the entire immune response against this antigen. In this context, IgG1 subtypes are more characteristic of a humoral response, associated with an increased secretion of interleukins IL-4 and IL-10 by activated lymphocytes, an increased level of subtype IgG2a is typical for a cellular Th1 response, associated with an increased secretion of IFNg and IL-12. The presence of the subtypes is not exclusive in this context, however the relative titers can be used as an indicator for the dominant type of the immune response that was formed.

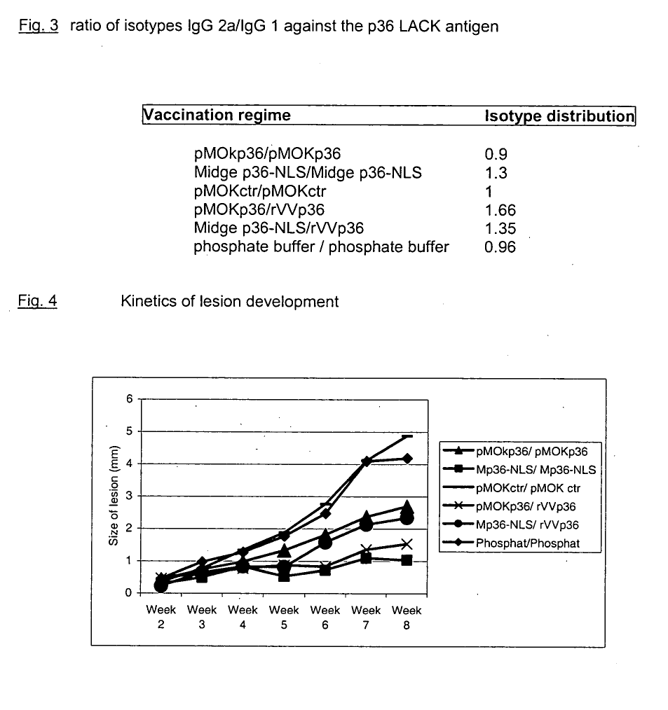

As is seen in FIG. 3, MIDGE vectors linked to the NLS peptide are capable of triggering a cellular (Th1) immune response. As was set out before, the cellular arm of the immune response is decisive in combating of intracellular parasites. The shift induced by MIDGE p36-NLS of the Th2 response towards a Th1 response is only marginally different from that induced by pMOKp36/rVVp36.

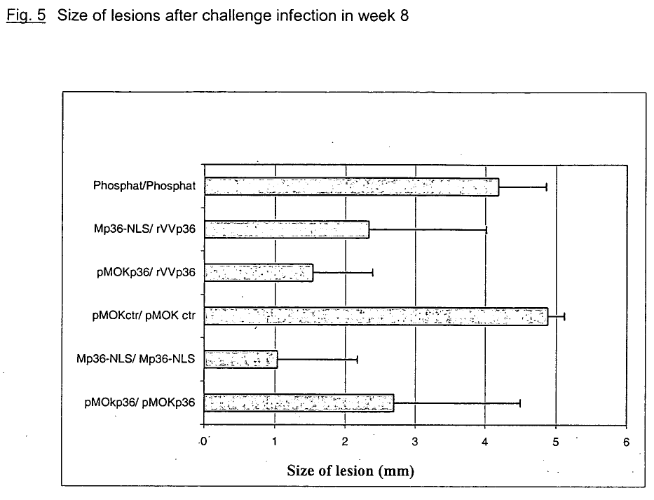

In order to assess the protection conferred, a challenge infection was performed using leishmania major promastigotes. Success of vaccination was rated in accordance to the growth progression of the lesions. It could be observed that the mice treated with MIDGE p36-NLS/MIDGE p36-NLS showed the smallest lesions, indicating that the vaccination regime MIDGE p36-NLS/MIDGE p36-NLS confers the longest protection by vaccination (see FIGS. 4 and 5).

These results are very surprising insofar as the pharmaceutical composition according to at least one embodiment is better with regard to its protective effect, than the currently “best” known vaccination regime of secondary immunization (boost) with recombinant vaccinia virus (rVV) that is state of the art. Additionally, it avoids the possible side effects attributed to plasmids and attenuated virus, and is yet comparable in its protective effect (Gonzalo et al., Microbes and Infection:3 (9):701-711). While of similar or better protective effect, the pharmaceutical composition according to at least one embodiment avoids the potential side effects of plasmids and recombinant virus as its decisive advantage, it can be produced more simply, more cheaply and, additionally, the inventive composition is much safer.

### EXAMPLES

### Example 1.1

### Recombinant Construction of the Plasmid pMOKp36

2 fragments were amplified by PCR from the starter plasmid pSCp36:

1. PCR approx. 800 bp;

| | | |
|---|---|---|
| Primer: | left 5′-TTATATGGTACCATGAACATACGAGGGTCACCT, | (= Seq ID 6) |
| Primer: | right 5′-TTATATGAGCTCAGAAGACACGGACAGGGACCTCTTCCGTCG | (= Seq ID 7) |

2. PCR approx. 950 bp;

| | | |
|---|---|---|
| Primer: | left 5′-TTATATGGTACCATGAACATACGAGGGTCACCT, | (= Seq ID 8) |
| Primer: | right 5′-TTATATGAGCTCTTACTCGGCCGTCGGAGATGG | (= Seq ID 9) |

The PCR product derived from the second PCR reaction was digested by Eco31I and the smaller fragment (approx. 200 bp) was isolated.

The PCR product from the first PCR reaction was digested with BpiI.

The 200 bp fragment and the digested fragment from the first PCR reaction were ligated and subsequently digested by KpnI and SacI, and inserted by ligation into the pMOK vector that had been digested by KpnI and SacI. The resulting plasmid was named pMOK p36. (=Seq ID 1).

### Example 1.2

### Covalent Attachment of the NLS Sequence to Oligonucleotides

Attachment of NLS was performed as follows: the NLS peptide comprising the sequence PKKKRKV was attached to the ODN in two steps. First, the modified oligonucleotide 5′-PH-dGGG AGT CCA GT xT TTC TGG AC (where xT represents an amino-modified thymine base with a C2 amino linking residue; =ODN 1=Seq ID 4) was activated with sulfo-KMUS (5 mM) in PBS at room temperature. The reaction was stopped after 120 min by adding 50 mM tris-(hydroxymethyl)-aminomethane and the activated ODN was obtained after ethanol precipitation (300 mM NaOAc pH 5.2, 5.5 mM MgCl₂, 70% ethanol) and a single round of washing with 70% ethanol. The ODN thus obtained was dissolved in PBS at 0.1 mM and reacted with the activated peptide (0.2 mM) for one hour at room temperature. The reaction was checked by gel electrophoresis and ethidium bromide staining. The NLS-attached ODN was purified by HPLC and used for the synthesis of MIDGE p36-NLS constructs.

### Example 1.3

### Production of MIDGE p36-NLS

MIDGE are linear covalently closed expression cassettes that only consist of the CMV promoter, an intron, the respective gene sequence and a polyadenylation sequence (see EP 0 941 318 B1). The constructs were obtained as follows: the plasmid pMOK p36 as described in example 1.1 was digested to completion by Eco31I. Ligation with 5′ phosphorylated hairpin-shaped 5′-PH-GGG AGT CCA GT XT TTC TGG AC (=ODN 1=Seq ID 4) and 5′-AGG GGT CCA GTT TTC TGG AC-3′ (=ODN 2=Seq ID 5), was achieved using T4 DNA ligase in the presence of Eco31 I, and stopped by heating to 70° C. The resulting mix was concentrated and treated with Eco31I and T7 DNA polymerase in the absence of deoxyribonucleotide trisphosphates. Purification was performed by anion exchange chromatography.

### Example 1.4

### Determination of 136 Antibody in Mice

MIDGE p36-NLS, pMOKp36 and recombinant vaccinia virus p36 (rVV) were injected into female mice (Balb/c) according to the following protocol.

TABLE 1

| | primary | Secondary immunization |
|---|---|---|
| group | immunization | (boost) |
| 1 | pMOKp36 | pMOKp36 |
| 2 | MIDGE p36-NLS | MIDGE p36-NLS |
| 3 | pMOK control | pMOK control |
| 4 | pMOK p36 | rVV p36 |
| 5 | MIDGE p36-NLS | rVV p36 |
| 7 | phosphate buffer | phosphate buffer |

10 mice were used per group.

Amounts of DNA were:

pMOK p36: 100 μg, i.d.

MIDGE p36-NLS: 54.8 μg, i.d.

rVV p36: 5×10⁷ pfu/animal, i.p.

and were applied dissolved in sodium phosphate buffer at pH 7.2.

After 2 weeks, the secondary immunization (boost) was performed with the respective DNA construct (see table 1). Three weeks after the boost, challenge infection was performed with 5×10⁴ leishmania major promastigotes. These were injected into the right hind paw subcutaneously. The state of infection was inspected weekly. The size of the lesions was determined using an electronic sliding calliper in comparison to the untreated left hind paw.

Eight weeks after the challenge infection, all mice were bled for sera. Determination of total IgG antibody titer against p36 and the determination of IgG 2a and IgG 1 was performed by means of ELISA, reading absorption as optical density at a wavelength of λ=406 nm.

One feature or aspect of an embodiment is believed at the time of the filing of this patent application to possibly reside broadly in a DNA expression construct for immunization against infections by leishmania, characterized by the immunizing polynucleotide sequences having the form of expression constructs consisting of covalently closed linear deoxyribonucleotide molecules, which comprise a linear double stranded region, the single strands forming said double stranded region being linked by short single stranded loops of deoxyribonucleic acid nucleotides, said double strand forming single strands consisting only of the coding sequence under control of a promoter and a terminator sequence operable in the animal that is to be vaccinated, and the DNA expression construct being covalently linked to one or more oligopeptides for increasing of the transfection efficacy.

Another feature or aspect of an embodiment is believed at the time of the filing of this patent application to possibly reside broadly in the DNA expression construct, where the expression construct encodes one or more leishmania antigens.

Yet another feature or aspect of an embodiment is believed at the time of the filing of this patent application to possibly reside broadly in the. DNA expression construct, where the expression construct encodes the p36 LACK antigen.

Still another feature or aspect of an embodiment is believed at the time of the filing of this patent application to possibly reside broadly in the DNA expression construct, where the oligopeptide consists of 3 to 30 amino acids, at least half of which are members of the group consisting of arginine and lysine.

A further feature or aspect of an embodiment is believed at the time of the filing of this patent application to possibly reside broadly in the DNA expression construct, where the oligopeptide comprises the amino acid sequence PKKKRKV (proline-lysine-lysine-lysine-arginine-lysine-valine).

Another feature or aspect of an embodiment is believed at the time of the filing of this patent application to possibly reside broadly in a Use of the DNA expression construct for the production of a vaccine for the treatment of leishmaniasis infectious diseases.

Yet another feature or aspect of an embodiment is believed at the time of the filing of this patent application to possibly reside broadly in the Vaccine for the treatment of leishmaniasis infectious diseases containing the DNA expression construct.

The components disclosed in the various publications, disclosed or incorporated by reference herein, may possibly be used in possible embodiments of the present invention, as well as equivalents thereof.

The purpose of the statements about the technical field is generally to enable the Patent and Trademark Office and the public to determine quickly, from a cursory inspection, the nature of this patent application. The description of the technical field is believed, at the time of the filing of this patent application, to adequately describe the technical field of this patent application. However, the description of the technical field may not be completely applicable to the claims as originally filed in this patent application, as amended during prosecution of this patent application, and as ultimately allowed in any patent issuing from this patent application. Therefore, any statements made relating to the technical field are not intended to limit the claims in any manner and should not be interpreted as limiting the claims in any manner.

The appended drawings in their entirety, including all dimensions, proportions and/or shapes in at least one embodiment of the invention, are accurate and are hereby included by reference into this specification.

The background information is believed, at the time of the filing of this patent application, to adequately provide background information for this patent application. However, the background information may not be completely applicable to the claims as originally filed in this patent application, as amended during prosecution of this patent application, and as ultimately allowed in any patent issuing from this patent application. Therefore, any statements made relating to the background information are not intended to limit the claims in any manner and should not be interpreted as limiting the claims in any manner.

Some examples of methods of and devices for performing a polymerase chain reaction (PCR) which may possibly be utilized in at least one possible embodiment may possibly be found in the following U.S. Pat. Nos.: 6,596,492; 6,586,250; 6,586,233; 6,569,678; 6,569,627; 6,566,067; 6,566,052; 6,558,929; 6,558,909; 6,551,783; 6,544,782; 6,524,830; 6,518,020; 6,514,750; 6,514,706; 6,503,750; 6,493,640; 6,492,114; 6,485,907; and 6,485,903.

All, or substantially all, of the components and methods of the various embodiments may be used with at least one embodiment or all of the embodiments, if more than one embodiment is described herein.

The purpose of the statements about the object or objects is generally to enable the Patent and Trademark Office and the public to determine quickly, from a cursory inspection, the nature of this patent application. The description of the object or objects is believed, at the time of the filing of this patent application, to adequately describe the object or objects of this patent application. However, the description of the object or objects may not be completely applicable to the claims as originally filed in this patent application, as amended during prosecution of this patent application, and as ultimately allowed in any patent issuing from this patent application. Therefore, any statements made relating to the object or objects are not intended to limit the claims in any manner and should not be interpreted as limiting the claims in any manner.

All of the patents, patent applications and publications recited herein, and in the Declaration attached hereto, are hereby incorporated by reference as if set forth in their entirety herein.

The summary is believed, at the time of the filing of this patent application, to adequately summarize this patent application. However, portions or all of the information contained in the summary may not be completely applicable to the claims as originally filed in this patent application, as amended during prosecution of this patent application, and as ultimately allowed in any patent issuing from this patent application. Therefore, any statements made relating to the summary are not intended to limit the claims in any manner and should not be interpreted as limiting the claims in any manner.

Some examples of the restriction enzyme Eco31I which may possibly be utilized in at least one possible embodiment may possibly be found in the following U.S. Pat. Nos.: 6,599,703; 6,579,705; 6,451,563; 6,344,345; 6,303,308; 6,258,533; 6,190,889; 5,858,671; 5,658,736; 5,468,851; 5,436,150; 5,356,802; and 5,278,051.

It will be understood that the examples of patents, published patent applications, and other documents which are included in this application and which are referred to in paragraphs which state “Some examples of . . . which may possibly be used in at least one possible embodiment of the present application . . . ” may possibly not be used or useable in any one or more embodiments of the application.

The sentence immediately above relates to patents, published patent applications and other documents either incorporated by reference or not incorporated by reference.

Some examples of methods of and devices for performing gel electrophoresis which may possibly be utilized in at least one possible embodiment may possibly be found in the following U.S. Pat. Nos.: 6,582,574; 6,576,104; 6,569,306; 6,535,624; 6,406,602; 6,379,515; 6,301,377; 6,258,544; 6,197,173; 6,190,522; 6,127,134; 6,057,106; 6,043,025; 6,001,233; 5,989,400; 5,972,188; 5,938,909; 5,938,906; 5,916,427; and 5,904,826.

All of the patents, patent applications or patent publications, which were cited in the international search report dated Jul. 2, 2003, and/or cited elsewhere are hereby incorporated by reference as if set forth in their entirety herein as follows: GONZALEZ-ASEGUINOLAZA, ET AL., “Molecular cloning, cell localization and binding affinity to DNA replication proteins of the p36/LACK protective from Leishmania infantum,” EUROPEAN JOURNAL OF BIOCHEMISTRY, Bd. 259, Nr. 3, February 1999 (1999-02), pages 909-916; MARIA-GONZALO, ET AL., “Protective immune response against cutaneous leishmaniasis by prime/booster immunization regimens with vaccinia virus recombinants expressing Leishmania infantum p36/LACK and IL-12 in combination with purified p36,” MICROBES AND INFECTION, Bd. 3, Nr. 9, July 2001 (2001-07), pages 701-711; WO 98 21322 A (JUNGHANS CLAAS; SOFT GENE GMBH (DE); WITTIG BURGHARDT (DE)) 22 May 1998 (1998 May 22); and LANFORD, ET AL., “Induction of Nuclear Transport with a Synthetic Peptide Homologous to the Sv-40 T Antigen Transport Signal,” CELL, Bd. 46, Nr. 4, 1986, pages 575-582.

The corresponding foreign and international patent publication applications, namely, Federal Republic of Germany Patent Application No. 101 48 732.0, filed on Oct. 2, 2001, having inventors Laura FUERTES-LÓPEZ and Marcos TIMÓN-JIMENÉZ, and DE-OS 101 48 732.0 and DE-PS 101 48 732.0, and Federal Republic of Germany Patent Application No. 101 56 679.4, filed on Nov. 12, 2001, having inventors Laura FUERTES-LÓPEZ and Marcos TIMÓN-JIMENÉZ, and DE-OS 101 56 679.4 and DE-PS 101 56 679.4, and International Application No. PCT/DE02/03799, filed on Oct. 2, 2002, having WIPO Publication No. WO03/031470 and inventors Laura FUERTES-LÓPEZ and Marcos TIMÓN-JIMENÉZ, as well as their published equivalents, and other equivalents or corresponding applications, if any, in corresponding cases in the Federal Republic of Germany and elsewhere, and the references and documents cited in any of the documents cited herein, such as the patents, patent applications and publications, are hereby incorporated by reference as if set forth in their entirety herein.

Some examples of methods of and devices for performing gene or genetic injection which may possibly be utilized in at least one possible embodiment may possibly be found in the following U.S. Pat. Nos.: 6,525,030; 6,361,991; 6,090,790; 5,998,382; 5,697,901; 5,661,133; 5,273,525; 6,482,405; and 6,063,629.

All of the references and documents, cited in any of the documents cited herein, are hereby incorporated by reference as if set forth in their entirety herein. All of the documents cited herein, referred to in the immediately preceding sentence, include all of the patents, patent applications and publications cited anywhere in the present application.

The following U.S. patent applications are hereby incorporated by reference as if set forth in their entirety herein: Ser. No. 10/057,311, filed Jan. 24, 2002, entitled “Covalently Closed Nucleic Acid Molecules for Immunostimulation,” and having inventors Junghans, et al. and attorney docket no. NHL-NP-37; Ser. No. 10/041,672, filed Jan. 8, 2002, entitled “Feline Interleukin-12 as Immunostimulant,” and having inventors Lutz, et al. and attorney docket no. NHL-NP-36; and Ser. No. ______, filed Apr. 1, 2004, entitled “Means for eliciting an immune response and a method therefor,” and having inventors Sonia MORENO-LÓPEZ and Marcos TIMÓN-JIMENÉZ and attorney docket no. NHL-NP-45,

The description of the embodiment or embodiments is believed, at the time of the filing of this patent application, to adequately describe the embodiment or embodiments of this patent application. However, portions of the description of the embodiment or embodiments may not be completely applicable to the claims as originally filed in this patent application, as amended during prosecution of this patent application, and as ultimately allowed in any patent issuing from this patent application. Therefore, any statements made relating to the embodiment or embodiments are not intended to limit the claims in any manner and should not be interpreted as limiting the claims in any manner.

The following U.S. patents may possibly disclose structures or processes that may possibly be used in at least one possible embodiment of the present invention, and are hereby incorporated by reference as follows: U.S. Pat. No. 6,534,271, issued to Furste, et al. on Mar. 18, 2003; U.S. Pat. No. 6,451,593 issued to Wittig, et al. on Sep. 17, 2002; and U.S. Pat. No. 6,451,563 issued to Wittig, et al. on Sep. 17, 2002.

The details in the patents, patent applications and publications may be considered to be incorporable, at applicant's option, into the claims during prosecution as further limitations in the claims to patentably distinguish any amended claims from any applied prior art.

The following U.S. patents and foreign patent publications may possibly disclose structures or processes that may possibly be used in at least one possible embodiment of the present invention, as follows: U.S. Pat. No. 5,580,859; U.S. Pat. No. 5,584,807; U.S. Pat. No. 5,589,466; DE 198 54 946; DE 196 48 625; DE 198 26 758; EP 0686697; EP 0732395; WO 9626270; WO 9632473; WO 92/13963; WO 9313216; WO 94/12633; and WO 98/21322.

The purpose of the title of this patent application is generally to enable the Patent and Trademark Office and the public to determine quickly, from a cursory inspection, the nature of this patent application. The title is believed, at the time of the filing of this patent application, to adequately reflect the general nature of this patent application. However, the title may not be completely applicable to the technical field, the object or objects, the summary, the description of the embodiment or embodiments, and the claims as originally filed in this patent application, as amended during prosecution of this patent application, and as ultimately allowed in any patent issuing from this patent application. Therefore, the title is not intended to limit the claims in any manner and should not be interpreted as limiting the claims in any manner.

The following foreign patent publications are hereby incorporated by reference as if set forth in their entirety herein: International Application No. PCT/DE02/03798 filed Oct. 2, 2002 and corresponding publication WO03/031469 published Apr. 17, 2003, Federal Republic of Germany Patent Application No. 101 48 697.9 filed Oct. 2, 2001, and Federal Republic of Germany Patent Application No. 101 56 678.6 filed Nov. 12, 2001.

The abstract of the disclosure is submitted herewith as required by 37 C.F.R. §1.72(b). As stated in 37 C.F.R. §1.72(b):

A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims.

Therefore, any statements made relating to the abstract are not intended to limit the claims in any manner and should not be interpreted as limiting the claims in any manner.

The following publications discuss genetic technology, such as gene therapy, DNA cloning, production, and manipulation thereof, and treatment and immunization of cells with DNA, and may possibly disclose structures or processes that may possibly be used in at least one possible embodiment of the present invention. These publications are incorporated by reference as follows: Eck, et al., 1996. Goodman & Gilman's The Pharmacological Basis of Therapeutics, Ninth Edition. McGraw-Hill, N.Y.; Johnston, et al., 1993. Genetic Engineering, 15:225-236; “Immunization by Direct DNA Inoculation Induces Rejection of Tumor Cell Challenge” Wang et al., Human Gene Therapy 6:407-418 (April 1995); “Identification of Wild-Type and Mutant p53 Peptides Binding to HLA-A2 Assessed by a Peptide Loading-Deficient Cell Line Assay and Novel Major Histocompatibilty Complex Class I Peptide Binding Assay” Stuber et al., Eur. J. Immunol. 1994. 24:765-768; “Particle-Mediated Gene Transfer of Granulocyte-Macrophage Colony-Stimulating Factor cDNA to Tumor Cells: Implications for a Clinically Relevant Tumor Vaccine” Mahvi et al., Human Gene Therapy 7:1535-1543 (Aug. 20, 1996); “Ex Vivo Regulation of Specific Gene Expression by Nanomolar Concentration of Double-Stranded Dumbbell Oligonucleotides” Clusel et al., Nucleic Acids Research, 1993, vol. 21, No. 15, 3405-3411; “Dendritic Cells as Initiators of Tumor Immune Responses: A Possible Strategy for Tumor Immunotherapy?” Grabbe et al., Immunology Today, vol. 16, No. 3 1995, 117-121; “Sequence-independent Inhibition of RNA Transcription by DNA Dumbbells and Other Decoys” Lim et al., Nucleic Acids Research, 1997, vol. 25, No. 3, 575-581; “A New Peptide Vector for Efficient Delivery of Oligonucleotides into Mammalian Cells” Morris et al., Nucleic Acids Research, 1997, vol. 25, No. 14, 2730-2736; “Improved Biological Activity of Antisense Oligonucleotides Conjugated to a Fusogenic Peptide” Bongartz et al., Nucleic Acids Research, 1994, vol. 22, No. 22, 4681-4688; “The Influence of Endosome-Disruptive Peptides on Gene Transfer Using Synthetic Virus-Like Gene Transfer Systems” Plank et al., The Journal of Biological Chemistry, vol. 269, No. 17, Apr. 29, pp. 12918-12924, 1994; “Linear Mitochondrial DNAs of Yeasts: Closed-Loop Structure of the Termini and Possible Linear-Circular Conversion Mechanisms” Dinouel et al., Molecular and Cellular Biology, April 1993, pp. 2315-2323; “Heterologous Protection Against Influenza by Injection of DNA Encoding a Viral Protein” Ulmer et al., Science, vol. 259, Mar. 19, 1993, pp. 1745-1749; “Comparison of Organic Monolayers on Polycrystalline Gold Spontaneously Assembled from Solutions Containing Dialkyl Disulfides or Alkanethiols” Biebuyck et al., Langmuir 1994, 10, 1825-1831; “Regression of Established Murine Carcinoma Metastases Following Vaccination with Tumour-Associated Antigen Peptides” Mandelboim et al., Nature Medicine, vol. 1, No. 11, November 1995, pp. 1179-1183; Kilisch et al. Covalently linked sequencing primer linkers (slinkers) for sequence analysis of restriction fragments. Gene vol. 44, pp. 263-270, December 1986; Roberts, RJ. Restriction and modification enzymes and their recognition sequences. vol. 13 Suppl. r165-r200, December 1985; and Berger and Kimmel. Guide to molecular cloning techniques. Methods in Enzymology. vol. 52, Academic Press, Inc. New York. pp. 307-661, December 1987.

The embodiments of the invention described herein above in the context of the preferred embodiments are not to be taken as limiting the embodiments of the invention to all of the provided details thereof, since modifications and variations thereof may be made without departing from the spirit and scope of the embodiments of the invention.

What is claimed is:

1. DNA expression construct for immunization against infections by leishmania, characterized by the immunizing polynucleotide sequences having the form of expression constructs consisting of covalently closed linear deoxyribonucleotide molecules, which comprise a linear double stranded region, the single strands forming said double stranded region being linked by short single stranded loops of deoxyribonucleic acid nucleotides, said double strand forming single strands consisting only of the coding sequence under control of a promoter and a terminator sequence operable in the animal that is to be vaccinated, and the DNA expression construct being covalently linked to one or more oligopeptides for increasing of the transfection efficacy.

2. DNA expression construct according to claim 1, where the expression construct encodes one or more leishmania antigens.

3. DNA expression construct according to claim 1, where the expression construct encodes the p36 LACK antigen.

4. DNA expression construct according to claim 1, where the oligopeptide consists of 3 to 30 amino acids, at least half of which are members of the group consisting of arginine and lysine.

5. DNA expression construct according to claim 4, where the oligopeptide comprises the amino acid sequence PKKKRKV (proline-lysine-lysine-lysine-arginine-lysine-valine).

6. Use of the DNA expression construct according to claim 1 for the production of a vaccine for the treatment of leishmaniasis infectious diseases.

7. Vaccine for the treatment of leishmaniasis infectious diseases containing the DNA expression construct according to claim 1.

8. DNA expression construct according to claim 2, where the expression construct encodes the p36 LACK antigen.

9. Use of the DNA expression construct according to claim 2 for the production of a vaccine for the treatment of leishmaniasis infectious diseases.

10. Use of the DNA expression construct according to claim 3 for the production of a vaccine for the treatment of leishmaniasis infectious diseases.

11. Use of the DNA expression construct according to claim 4 for the production of a vaccine for the treatment of leishmaniasis infectious diseases.

12. Use of the DNA expression construct according to claim 5 for the production of a vaccine for the treatment of leishmaniasis infectious diseases.

13. Use of the DNA expression construct according to claim 8 for the production of a vaccine for the treatment of leishmaniasis infectious diseases.

14. Vaccine for the treatment of leishmaniasis infectious diseases containing the DNA expression construct according to claim 2.

15. Vaccine for the treatment of leishmaniasis infectious diseases containing the DNA expression construct according to claim 3.

16. Vaccine for the treatment of leishmaniasis infectious diseases containing the DNA expression construct according to claim 4.

17. Vaccine for the treatment of leishmaniasis infectious diseases containing the DNA expression construct according to claim 5.

18. Vaccine for the treatment of leishmaniasis infectious diseases containing the DNA expression construct according to claim 8.
